(19) United States
(12) Patent Application Publication
(10) Pub. No.: US 2005/0037063 A1
(43) Pub. Date: Feb. 17, 2005

(54) Combined therapies

(76) Inventors: Anthony E. Bolton, Santry (IE); Arkady Mandel, North York (CA)

Correspondence Address: FOLEY & LARDNER LLP;Three Palo Alto Square, Suite 100, 3000 El Camino Real, Palo Alto, CA, 94306, US

(21) Appl. No.: 10/893,113

(22) Filed: Jul. 16, 2004

Related U.S. Application Data
(60) Provisional application No. 60/489,069, filed on Jul. 21, 2003.

(51) Int. Cl. A61K 9/127

(52) U.S. Cl. 424/450

(57) ABSTRACT

This invention provides a combination treatment and compositions for alleviating the symptoms of a cardiovascular disorder in a mammalian patient, which comprises administration to the patient of an effective amount of synthetic and/or semi-synthetic bodies carrying phospholipid ligands on their surfaces, said bodies have a size of from about 20 nanometers to about 500 microns; and an effective amount of a cardiovascular drug.

### CROSS-REFERENCE TO RELATED APPLICATIONS

This application claims the benefit under 35 U.S.C. §119(e) of U.S. Provisional Patent Application Ser. No. 60/489,069, filed Jul. 21, 2003 which is incorporated herein be reference in its entirety.

### FIELD OF THE INVENTION

This invention relates to compositions and procedures for the treatment of cardiovascular disorders such as angina, arrhythmia, hypertension and hyperlipidemia in mammalian patients.

### BACKGROUND OF THE INVENTION

Hyperlipidemias such as hypercholesterolemia and elevated serum triglyceride levels are among the most potent risk factors in the causation of atherosclerosis, which is the build-up of fatty plaque deposits within the walls of blood vessels. For example, high levels of serum cholesterol bound to low density lipoprotein (LDL), intermediate density lipoprotein (IDL) or very low density lipoprotein (VLDL) are known to correlate strongly with the occurrence of atherosclerosis in humans. In particular it is known that the higher the levels of circulating cholesterol in the form of LDL, IDL and VLDL cholesterol, and the higher the levels of other lipids such as triglycerides, the more likely it is that cholesterol and lipids will be deposited within the blood vessel wall and cause or contribute to atherosclerosis.

Reduction of hyperlipidemia, including hypercholesterolemia, results in delayed onset of atherosclerosis and a decrease in progression of atherosclerosis, thus reducing the risk of coronary heart disease. Atherosclerosis, the major cause of most cardiovascular disorders, is a complex process involving the blood vessel wall, many different blood elements and the immune system. Recent evidence indicates that immune-related inflammation of the TH1 type is a major factor in the initiation, progression and manifestations of the disease. Down-regulation of the disease has been shown to prevent the development of, and/or cause regression of, atherosclerosis.

High blood pressure or hypertension is one of the most common cardiovascular diseases and refers to a condition in which blood pressure is persistently elevated. When blood pressure is elevated for an extended period of time, the inner linings of the coronary arteries become damaged. This leaves them susceptible to the buildup of fatty deposits that can narrow or block the arteries and reduce blood flow to the body's organs. When untreated, high blood pressure can lead to heart failure, stroke, kidney damage, and loss of vision from damage to the retina at the back of the eye. Hypertension is classified by etiology as being either primary (essential, idiopathic) in 90-95% of cases or secondary. Primary hypertension has no clearly defined etiology but some of the potential factors include sympathetic nervous system dysfunction and/or hyperactivity, renin-angiotensin system defects, sodium transport defects, intracellular sodium and calcium and other factors such as obesity, excessive alcohol use, cigarette smoking, excessive salt intake, etc. Hypertension is the most important etiologic factor for cardiovascular disease in the US. Cardiovascular and atherosclerotic complications are the major cause of morbidity and mortality in patients with primary hypertension. Hypertension is also a major cause of stroke as it increases the risk of cerebral vascular insufficiency.

Any variation from the normal rhythm and sequence of excitation of the heart is called an arrhythmia. Arrhythmia may result from many factors, including interference with conduction of electrical signals, ectopic foci, alterations in the activity of the sinoatrial (SA) node, acute ischemia (oxygen deprivation), SNS activation, and myocardial scarring.

Variant angina pectoris is also known as Prinzmetal's angina. It differs from typical angina in that it occurs almost exclusively when a person is at rest. Attacks can be very painful and usually occur between midnight and 8 a.m. Variant angina pectoris, like other forms of angina and anginal equivalents, can be associated with acute myocardial infarction, severe cardiac arrhvthmias, including ventricular tachycardia, fibrillation, and even sudden death. Variant angina is due to coronary artery spasms, which can occur in close proximity to an athersclerotic obstruction. Many of the people with angina go through an acute active phase. Anginal and cardiac events may occur frequently for six months or more. Angina pectoris is defined as chest pain or discomfort of cardiac origin often radiating from the precordium to the left shoulder and down the left arm, that usually results from a temporary imbalance between myocardial oxygen supply and myocardial oxygen demand. The discomfort is often induced by exercise, emotion, eating, or cold weather. Pain is more likely to occur when the subject is outdoors, especially when the temperature is extremely high or low and when the patient is walking uphill against the wind. Angina commonly occurs when a subject has eaten a heavy meal or when the subject is excited, angry or tense. Often angina pectoris is due to ischemia of the heart and is usually caused by coronary disease.

Congestive heart failure (CHF) is a relatively common disorder affecting approximately five million Americans, with a mortality rate of over 80,000 per year. It is believed that CHF is not a distinct disease process itself, but rather represents the effect of multiple anatomic, functional and biologic abnormalities which interact to ultimately produce progressive loss of ability of the heart to fulfil its function as a circulatory pump. CHF may be caused by occurrence of an event such as myocardial infarction or be secondary to other causes such as hypertension or cardiac malformations such as valvular disease. Some of the more important pathophysiological changes which occur in CHF are activation of the hypothalamic-pituitary-adrenal axis, systemic endothelial dysfunction and myocardial remodelling.

Atherosclerosis is the build-up of fatty acid plaque deposits within the walls of blood vessels, commonly develops over a relatively lengthy period of time in patients. To-date, treatments such as diet adjustment and administration of cholesterol-lowering drugs have slowed or even halted the development of atherosclerosis, but only limited success has been reported for causing the regression of atherosclerosis i.e. diminution of the atherosclerotic fatty plaque deposits. The need for procedures and compositions which will not only reduce the serum levels, especially cholesterol levels, in mammalian patients, but will also inhibit the development and effect the regression of atherosclerotic plaque deposits and/or delay or ameliorate one or more symptoms associated with atherosclerosis and the aforementioned cardiovascular disorders, is clearly apparent.

Drugs are available and are widely prescribed for the treatment of the aforementioned cardiovascular disorders. For hypercholesterolemia, there are various statin drugs such as atorvastatin. For hypertension, a wide variety of anti-hypertensive drugs such as angiotensin converting enzyme inhibitors (ACE inhibitors) e.g. enalapril are available. For arrhythmia, there are anti-arrhythmia drugs such as digitalin and calcium channel blockers such as nifedipine. For angina, there are various categories of anti-anginal drugs such as beta-blockers (e.g. betaxolol) and vasodilators such as nitroglycerine.

There are undesirable side effects associated with many cardiovascular drugs. The risks associated with long term administration of such drugs are in many cases unknown. Since for the most part they are synthetic chemical materials, it is desirable to limit their usage, and reduce dosage amounts, to reduce potential long term side effects.

Therapies specifically directed at counteracting the activation of the hypothalamic-pituitary-adrenal axis (beta-adrenergic blockers such as bisoprolol, metoprolol, atenolol etc., ACE inhibitors captopril, ramipril, enalapril, lisinopril, quinopril and ramipril prepared as described above, certain calcium channel blockers, nitrates, diuretics, both loop and potassium sparing and endothelin-1 blocking agents) have been used in the treatment of CHF. Aldosterone agents such a spironolactone (U.S. Pat. No. 3,257,390) have also been used.

Bolton et al, United States Publication Nos. US 2002-0086049, US 2003-0175334, and 2004-0013718 (herein incorporated by reference in their entirety), describe synthetic and semi-synthetic three-dimensional bodies carrying anionic and other phospholipid ligands on their surfaces, and administration to the patient of these bodies for alleviation of a variety of disorders where T-cell function, inflammation, endothelial dysfunction and inappropriate cytokine expression are involved.

In view of the above, it would be beneficial to provide a prophylactic or therapeutic treatment for cardiovascular disorders such as angina, arrhythmia, congestive heart failure, peripheral areterial occlusive disease (or peripheral vascular disease, PVD), hypertension and hyperlipidemia in mammalian patients, in which the benefits of the existing treatments are enhanced and/or in which the disadvantages associated with the existing treatments for this purpose are reduced.

### SUMMARY OF THE INVENTION

This invention is based upon the discovery that pharmaceutically acceptable bodies, such as liposomes, beads or similar particles, carrying anionic or other phospholipids such as phosphatidyl-glycerol, phosphatidyl-serine, phosphatidyl-inositol and the like, will, in combination with various cardiovascular drugs, on administration to a patient, cause an anti-inflammatory effect and therefore may be used to treat a variety of cardiovascular disorders, preferably those mediated, at least in part, by an inflammatory component.

One aspect of this invention provides a combination treatment for alleviating the symptoms of a cardiovascular disorder in a mammalian patient, which comprises administration to the patient of (a) an effective amount of synthetic and/or semi-synthetic bodies carrying phospholipid ligands on their surfaces, said bodies have a size of from about 20 nanometers to about 500 microns; and (b) an effective amount of a cardiovascular drug.

Another aspect of the invention provides a composition for treating a cardiovascular disorder in a mammalian patient, said composition comprising (a) an effective amount of synthetic and/or semi-synthetic bodies carrying phospholipid ligands on their surfaces, said bodies having a size of from about 20 nanometers to about 500 mcirons, and (b) an effective amount of cardiovascular drug.

Yet another aspect of the invention is the provision of a combination treatment for slowing or arresting the progression and/or effecting the regression of atherosclerotic plaque deposits in a mammalian patient by the reduction of cholesterol, for reducing blood pressure, prevention and/or treatment of arrhythmias, for the prevention and/or treatment of angina, for the treatment or inhibition of the progression of peripheral arterial occlusive disease, or for treatment or inhibition of progression of CHF, said combination treatment comprises the administration to a patient of synthetic and semi-synthetic bodies carrying anionic and other phospholipid ligands on their surfaces and having a size from about 20 nanometers (nm) to 500 microns (μm), and at least one drug selected from the therapeutic categories of anti-hypertensives, antianginals, antiarrythmias, antihyperlipoproteinemias, calcium channel blockers, cardiotonics, coronary vasodilators, peripheral vasodilators, cerebral vasodilators and diuretics.

Combination treatment with various cardiovascular drugs includes, for example, administration of the compositions described herein, prior to, during or after administration of one or more cardiovascular drugs. One of skill in the art will be able to determine the administration schedule and dosage. “Treatment” includes, for example, a reduction in the number of symptoms, a decrease in the severity of at least one symptom of the particular disease or a delay in the further progression of at least one symptom of the particular disease.

Another aspect of the invention comprises the use for slowing or arresting the progression and/or effecting the regression of atherosclerotic plaque deposits in a mammalian patient by the reduction of cholesterol, for reducing blood pressure, prevention and/or treatment of arrhythmias, for the prevention and/or treatment of angina, for the treatment or inhibition of the progression of peripheral arterial occlusive disease, or for treatment or inhibition of progression of CHF, of synthetic and semi-synthetic bodies carrying anionic and other phospholipid ligands on their surfaces and having a size from about 20 nanometers (nm) to 500 microns (μm), and at least one drug selected from the therapeutic categories of anti-hypertensives, antianginals, antiarrythmias, antihyperlipoproteinemias, calcium channel blockers, cardiotonics, coronary vasodilators, peripheral vasodilators, cerebral vasodilators and diuretics.

A further aspect of the invention is a process for enhancing the reduction in serum lipid levels (e.g., cholesterol or triglycerides) in a mammalian patient by administration of an antihyperlipoproteinemic drug and synthetic and semi-synthetic bodies carrying anionic and other phospholipid ligands on their surfaces and having a size from about 20 nanometers (nm) to 500 microns (μm). A further aspect of the invention is a process for enhancing the prevention and/or treatment of hypertension in a mammalian patient by administration of anti-hypertensive drugs and synthetic and semi-synthetic bodies carrying anionic and other phospholipid ligands on their surfaces and having a size form about 20 nanometers (nm) to 500 microns (μm). A further aspect of the invention is a process for enhancing the prevention and/or treatment of arrhythmia in a mammalian patient by administration of anti-arrhythmic drugs and synthetic and semi-synthetic bodies carrying anionic and other phospholipid ligands on their surfaces and having a size from about 20 nanometers (nm) to 500 microns (μm). A further aspect of the invention is a process for enhancing the prevention and/or treatment of angina in a mammalian patient by administration of anti-angina drugs and synthetic and semi-synthetic bodies carrying anionic and other phospholipid ligands on their surfaces and having a size from about 20 nanometers (nm) to 500 microns (μm). A further aspect of the invention is a process for retarding the progression of and treating CHF in a mammalian patient by administration of drugs for the treatment of CHF as previously described and synthetic and semi-synthetic bodies carrying anionic and other phospholipid ligands on their surfaces and having a size from about 20 nanometers (nm) to 500 microns (μm). A further aspect of the invention is a process for retarding the progression of and treating peripheral arterial occlusive disease in a mammalian patient by administration of drugs for the treatment of peripheral arterial occlusive disease as previously described and synthetic and semi-synthetic bodies carrying anionic and other phospholipid ligands on their surfaces and having a size from about 20 nanometers (nm) to 500 microns (μm). Such synthetic and semi-synthetic bodies are disclosed below and also found in, for example, Bolton et al., United States Publication Nos. US 2002-0086049, US 2003-0175334, and 2004-0013718 herein incorporated in their entirety by reference.

A further aspect of the present invention is a composition for treating one or more of the aforementioned cardiovascular disorders, said composition comprising an effective amount of synthetic and semi-synthetic bodies carrying anionic and other phospholipid ligands on their surfaces and having a size from about 20 nanometers (nm) to 500 microns (μm), and at least one drug selected from the therapeutic categories of anti-hypertensives, antianginals, antiarrythmias, antihyperlipoproteinemias, calcium channel blockers, cardiotonics, coronary vasodilators, peripheral vasodilators, cerebral vasodilators and diuretics.

### DESCRIPTION OF THE PREFERRED EMBODIMENTS

Antihypertensive drugs useful in the present invention include ACE-inhibitors, alpha-adrenergic agonists, beta-adrenergic agonists, alpha-adrenergic blockers, beta-adrenergic blockers and angiotensin II receptor antagonists.

Examples of ACE-inhibitors useful in the present invention include but are not limited to: alacepril, (U.S. Pat. No. 4,248,883); benazepril (U.S. Pat. No. 4,410,520); captopril (U.S. Pat. Nos. 4,046,889); ceronapril (U.S. Pat. No. 4,452,790); delapril, (U.S. Pat. No. 4,385,051); enalapril (U.S. Pat. No. 4,374,829); fosinopril (U.S. Pat. No. 4,337,201); imadapril (U.S. Pat. No. 4,508,727); lisinopril (U.S. Pat. No. 4,555,502); moveltopril (Belgian Patent No. 893,553); perindopril, (U.S. Pat. No. 4,508,729); quinapril (U.S. Pat. No. 4,344,949); ramipril (U.S. Pat. No. 4,587,258); spirapril (U.S. Pat. No. 4,470,972); temocapril (U.S. Pat. No. 4,699,905); and trandolapril (U.S. Pat. No. 4,933,361). Each of these patents is incorporated herein by reference in its entirety.

Alpha-adrenergic receptor blockers useful in this invention include, but are not limited to: amosulalol (U.S. Pat. No. 4,217,307); arofinolol (U.S. Pat. No. 3,932,400); dapiprazole (U.S. Pat. No. 4,252,721); doxazosin (U.S. Pat. No. 4,188,390); fenspiride (U.S. Pat. No. 3,399,192); indoramin, (U.S. Pat. No. 3,527,761); labetolol; naftopidil (U.S. Pat. No. 3,997,666); nicergoline (U.S. Pat. No. 3,228,943); prazosin (U.S. Pat. No. 3,511,836); tamsulosin (U.S. Pat. No. 4,703,063); tolazoline (U.S. Pat. No: 2,161,938); trimazosin (U.S. Pat. No. 3,669,968); and yohimbine, which may be isolated from natural sources according to methods well known to those skilled in the art. Each of these patents is incorporated herein by reference in its entirety.

Beta-adrenergic receptor blockers which are within the scope of this invention include, but are not limited to: acebutolol, (U.S. Pat. No. 3,857,952); alprenolol, befunolol (U.S. Pat. No. 3,853,923); betaxolol (U.S. Pat. No. 4,252,984); bevantolol (U.S. Pat. No. 3,857,981); bisoprolol (U.S. Pat. No. 4,171,370); bopindolol, (U.S. Pat. No. 4,340,541); bucumolol, (U.S. Pat. No. 3,663,570); buprandolol, (U.S. Pat. No. 3,309,406); bubridine hydrochloride, (French Patent No. 1,390,056); butofilolol, (U.S. Pat. No. 4,252,825); carteolol, (U.S. Pat. No. 31910,924); carvedilol, (U.S. Pat. No. 41503,067); cetamolol, (U.S. Pat. No. 4,059,622); cloranolol, (German Patent No. 2,213,044); epanolol, (European Patent Publication Application No. 41,491);. indenolol, (U.S. Pat. No. 4,045,482); labetalol, (U.S. Pat. No. 4,012,444); levobunolol, (U.S. Pat. No. 4,463,176); metoprolol, (U.S. Pat. No. 3,873,600); moprolol, (U.S. Pat. No. 3,501,7691); nadolol, (U.S. Pat. No. 3,935, 267); nadoxolol, (U.S. Pat. No. 3,819,702); nebivalol, (U.S. Pat. No. 4,654,362); nipradilol, (U.S. Pat. No. 4,394,382); oxprenolol, (British Patent No. 1,077,603); perbutolol, (U.S. Pat. No. 3,551,493); pindolol, (U.S. Pat. No. 3,471,515); pronethalol, (British Patent No. 909,357); propranolol, (U.S. Pat. Nos. 3,337,628 and 3,520,919) and timolol, (U.S. Pat. No. 3,655,663). Each of these patents and patent applications is incorporated herein by reference in its entirety.

One particularly preferred β-blocker drug for use in combination with the liposomes of the present invention is carbedilol, the full chemical name and chemical formula of which is 1-(9H-carbazol-4-ylosay)-3-[(2-(2-methoxypenoxy) ethyl] amino-2-propanol: 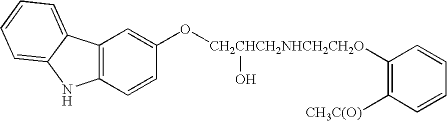
It is sold commercially, and is characterized as an antihypertensive, and in treatment of congestive heart failure.

Carvedilol possesses the combined properties of a β-adrenergic blocker and an antioxidant. This combination of properties, in conduction with the anti-inflammatory, immunoregulatory properties of the semi-synthetic or synthetic bodies carrying phospholipids, the other ingredient of the combinations of the present invention, provide a particularly attractive combination therapy, in treatment of cardiovascular disorder. Liposomes of phosphatidylglycerol, with external presentation of phosphate-glycerol active head groups, are preferred as the second active ingredient of the combination with carvedilol, especially such liposomes in which prhosphate glycerol constitutes 60-90% of the phospholipid external phosphor-containing groups.

The dosage of carvedilol in such a combination use is suitably from 0.1 mg to 10 mg per kilogram mammalian patient body weight, more preferably from 1 mg to 5 mg per kilogram. The liposomes are suitably used in a dosage of from about 500 to 2.5×10¹⁰, preferably from about 1000 to 1.5×10⁸, vesicles per dose, in such combinations.

Angiotensin-II receptor antagonists which can be used in this invention include, but are not limited to: candesartan, (U.S. Pat. No. 5,196,444); eprosartan, (U.S. Pat. No. 5,185,351); irbesartan, (U.S. Pat. No. 5,270,317); losartan, (U.S. Pat. No. 5,138,069); and valsartan, (U.S. Pat. No. 5,399,578). Each of these patents is incorporated herein by reference in its entirety.

Antianginal drugs useful in the present invention include but are not limited to beta blockers, calcium channel antagonists, vasodilators and lipid lowering drugs. The beta blocker can, by way of example, be one of those referenced above (acebutolol, alprenolol, befunolol, betaxolol, bevantolol, bisoprolol, bopindolol, bucumolol, buprandolol, bubridine hydrochloride, butofilolol, carteolol, carvedilol, cetamolol, cloranolol, epanolol, indenolol, labetalol, levobunolol, metoprolol, moprolol, nadolol, nadoxolol, nebivalol, nipradilol, oxprenolol, perbutolol, pindolol, pronethalol, propranolol, nitroglycerine and timolol).

Antiarrhythmia drugs useful in the present invention include digitalis (functions in slowing the electrical signal as it travels through the heart muscle to slow a rapid heartbeat or digoxin (U.S. Pat. No. 3,932,626, which is incorporated herein by reference in its entirety); calcium channel blockers, which reduce the force and rate of heart contraction, thus slowing a rapid heartbeat); beta blockers as described above, which, like digitalis, reduce the speed of heart contraction and therefore help to slow the heart rate; and anticoagulants, i.e. blood thinners which help to reduce the risk of blood clots and stroke caused by slow or weak heart contractions. Examples of suitable anticoagulants for use in the present invention include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, pentosan polysulfate, phenindione, phenprocoumon, phosvitin, picotamide, tioclomarol, and warfarin. Other medications that slow down rapid heart rhythms, and which can therefore be used in this invention, include quinidine, procainamide, disopyramide, flecainide, propafenone, sotalol, dofetilide, and amiodarone.

Antihyperlipoproteinemia drugs useful in the present invention include the so-called “statin” drugs such as lovastatin (U.S. Pat. No. 4,231,938); simvastatin (U.S. Pat. No. 4,444, 784); pravastatin (U.S. Pat. No. 4,346,227); fluvastatin (U.S. Pat. No. 4,739,073); mevastatin compactin, (U.S. Pat. No. 3,983,140); atorvastatin (U.S. Pat. Nos. 4,681,893 and 5,273,995); cerivastatin (5,502,199); dalvastatin (European Patent Publication 738,510 A2); velostatin (U.S. Pat. Nos. 4,448,784 and 4,450,171); and fluindostatin (European Patent Publication 363,934). Each of these patents and publications is incorporated herein by reference in its entirety. These are generally known as HMG-CoA reductase inhibitors, since their mechanism is understood to be through the selective, competitive inhibition of the hepatic enzyme 3-hydroxy-3-methaglutaryl-co-enzyme A. In some cases e.g. simvastatin, the active molecule is a metabolite formed in the body after ingestion of the drug by the patient. In other cases, the administered drug (commonly a pharmaceutically acceptable salt) is itself the active molecule. Particularly preferred among the statin drugs is atorvastatin calcium. This acts as an HMG-CoA reductase inhibitor to reduce serum cholesterol levels. It has also been reported to have beneficial effects in endothelial functions in sickle cell disease, and in halting the progression of heart disease. This combination of properties, in conjunction with the anti-inflammatory, immunoregulatory properties of the synthetic or semi-synthetic bodies carrying phospholipids, the other ingredient of the combinations of the present invention, provide a particularly attractive combination therapy, in treatment of cardiovascular disorder.

Other classes of cholesterol-lowering drugs include bile acid sequestrants (cholestyramine resin, colestipol, colesevelam hydrochloride and polidexide); fibrates (bezafibrate, binifibrate, ciprofibrate, clinofibrate, clofibrate, clofibric acid, etofibrate, fenofibrate, gemfibrozil, pirifibrate, ronifibrate, simfibrate and theofibrate); niacin derivatives (niacin and xanthinol niacinate); nicotinic acid derivatives (acipimox, aluminium nicotinate, niceritrol, nicoclonate, nicomol and oxiniacic acid, for example); thyroid hormone analogs such as dextrothyroxine, etiroxate and thyropropic acid); and other miscellaneous compounds such as acifran, benfluorex, beta-benzalbutyramide, carnitine, chondroitin sulfate, clomestrone, detaxtran, dextran sulfate sodium, eicosapentaenoic acid, eritadenine, ezetimide, furazabol, meglutol, dextrothyroxine, melinamide, gamma-oryzanol, pantethine, pentaerythritol tetraacetate, alpha-phenylbutyramide, pirozadil, probucol, beta-sitosterol, sultosilic acid, tiadenol, triparanol and xenbucin. All of these are useful in the present invention.

Calcium channel blockers which may be used in this invention include, but are not limited to: bepridil, (U.S. Pat. No. 3,962,238 or U.S. Reissue No. 30,577); clentiazem, (U.S. Pat. No. 4,567,175); diltiazem, (U.S. Pat. No. 3,562,257); fendiline, (U.S. Pat. No. 3,262,977); gallopamil, (U.S. Pat. No. 3,261,859); mibefradil, (U.S. Pat. No. 4,808,605); prenylamine, (U.S. Pat. No. 3,152,173); semotiadil, (U.S. Pat. No. 4,786,635); terodiline, (U.S. Pat. No. 3,371,014); verapamil, (U.S. Pat. No. 3,261,859); amlodipine (U.S. Pat. No. 4,572,909); aranipine, (U.S. Pat. No. 4,572,909); barnidipine, (U.S. Pat. No. 4,220,649); benidipin, (European Patent Application Publication No. 106,275); cilnidipine, (U.S. Pat. No. 4,672,068); efonidipine, (U.S. Pat. No. 4,885,284); elgodipine, (U.S. Pat. No. 4,952,592); felodipine, (U.S. Pat. No. 4,264,611); isradipine, (U.S. Pat. No. 4.466,972); lacidipine, (U.S. Pat. No. 41801,599); lercanidipine, (U.S. Pat. No. 4,7051797); manidipine, (U.S. Pat. No. 41892,875); nicardipine, (U.S. Pat. No. 30985,758); nifedipine, (U.S. Pat. No. 3,485,847); nilvadipine, (U.S. Pat. No. 4,338,322); nimodipine, (U.S. Pat. No. 3,799,934); nisoldipine, (U.S. Pat. No. 41154.839); nitrendipine, (U.S. Pat. No. 31799,934); cinnarizine, (U.S. Pat. No. 2,882,271); dotarizine, flunarizine, lidoflazine, lomerizine, bencyclane, etafenone, fantofarone, monatepil and perhexiline. Each of these patents and publications is incorporated herein by reference in its entirety.

Cardiotonics useful in the present invention include acetyldigitoxins, 2-amino-4-picoline, amrinone, benfurodil hemisuccinate, bucladesine, convallatoxin, cymarain, denopamine, deslanoside, digitalin, digitalis, digitoxin, digoxin, dobutamine, docarpamine, dopamine, dopexamine, enoximone, erythrophleine, gitalin, gitoxin, glycocyamine, heptaminol, hydrastinine, ibopamine, lanotosides, levosimendan, loprinone, milrinone, neriifolin, oleandrin, ouabin, oxyfedrin, pimobendan, prenalterol, proscillaridin, resibufogenin, scillaren, scillarenin, strophanthin, silmazole, theobromine, vesnarinone and xamoterol.

Coronary vasodilators useful in the present invention include but are not limited to: amotriphene, (U.S. Pat. No. 3,010,965); benfurodil hemisuccinate, (U.S. Pat. No. 3,355,463); benziodarone, (U.S. Pat. No. 3,012,042); chloracizine, (British Patent No. 740,932); chromonar); clobenfurol, (British Patent No. 1,160,925); clonitrate; cloricromen, (U.S. Pat. No. 4,452,811); dilazep, (U.S. Pat. No. 3,532,685); dipyridamole, (British Patent No. 807,826); droprenilamine, (German Patent No. 2,521,113); efloxate, (British Patent Nos. 803,372 and 824,547); erythrityl tetranitrate, which may be prepared by nitration of erythritol according to methods well-known to those skilled in the art); etafenone, (German Patent No. 1,265,758); fendiline, (U.S. Pat. No. 3,262,977); floredii, (German Patent No. 2,020,464); hexestrol, (U.S. Pat. No. 2,357,985); hexobendine, (U.S. Pat. No. 3,267,103); itramin tosylate; khellin; lidoflazine, (U.S. Pat. No. 3,267,104); mannitol hexanitrate; medibazine, (U.S. Pat. No. 3,119,826); nitroglycerine; pentaerythritol tetranitrate; pentrinitrol, (U.S. Pat. No. 3,419,571); perhexiline); pimefyiline, (U.S. Pat. No. 3,350,400); prenylamine, (U.S. Pat. No. 3,152,173); propatyl nitrate, (French Patent No. 1,103,113); trapidil, (East German Patent No. 55,956); tricromyl, (U.S. Pat. No. 2,769,015); trimetazidine, (U.S. Pat. No. 3,262,852); trolnitrate phosphate, which may be prepared by nitration of triethanolamine followed by precipitation with phosphoric acid according to methods well-known to those skilled in the art; and visnadine, (U.S. Pat. Nos. 2,816,118 and 2,980,699). Each of these patents is incorporated herein by reference in its entirety.

Cerebral vasodilators within the scope of this invention include, but are not limited to: citicoline, which may be isolated from natural sources as disclosed in Kennedy et al., Journal of the American Chemical Society, 1955, 77 250 or synthesized as disclosed in Kennedy, Journal of Biological Chemistry, 1956, 222, 185); cyclandelate, (U.S. Pat. No. 3,663,597); ciclonicate, (German Patent No. 1,910,481); diisopropylamine dichloroacetate, (British Patent No. 862,248); ebumamonine, Hermann et al., Journal of the American Chemical Society, 1979. 101, 1540; fasudil, (U.S. Pat. No. 4,678,783); fenoxedil, (U.S. Pat. No. 3,818,021); flunarizine, (U.S. Pat. No. 3,773,939); ibudilast, (U.S. Pat. No. 3,850,941); ifenprodil, (U.S. Pat. No. 3,509,164); lomerizine, (U.S. Pat. No. 4,663,325); nafronyl, (U.S. Pat. No. 3,334,096); nicametate, Blicke et al., Journal of the American Chemical Society, 1942, 64, 1722; vasodilators nicergoline, which may be prepared as disclosed above; nimodipine, papaverine, which may be prepared as reviewed in Goldberg, Chem. Prod. Chem. News, 1954, 17, 371; pentifylline, (German Patent No. 860,217); tinofedrine, (U.S. Pat. No. 3,563,997); vincamine, (U.S. Pat. No. 3,770,724); vinpocetine, (U.S. Pat. No. 4,035,750); and viquidil, (U.S. Pat. No. 2,500,444). Each of these patents and publications is incorporated herein by reference in its entirety.

One particularly preferred cerebral vasodilator drug for use in the combinations of the present invention is citicoline, the full chemical name and chemical formula of which is cytidine 5′-(trihydrogen diphosphate) p′-[2-(trimethylammonio)ethyl] ester inner salt, 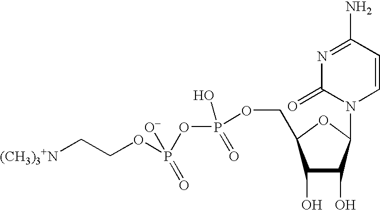
It can also be used in the form of its sodium salt. Citicoline occurs naturally in the mammalian body.

Citicoline has been reported to exert neuroprotective effects by increasing the levels of reduced glutathione (GSH), an ROS (reactive oxygen species)-scavenging enzyme in sections of the brain, by attenuating PLA-2 (phospholipase A-2) activity, activating tyrosine hydroxylase (TH) and increasing striatum-dopamine levels. This combination of properties, in conjuction with the anti-inflammatory, immunoregulatory properties of the semi-synthetic or synthetic bodies carrying phospholipids, the other ingredient of the combinations of the present invention, provide a particularly attractive combination therapy.

Liposomes of phosphatidylglycerol, with external presentation of phosphate-glycerol active head groups, are preferred as the second active ingredient of the combination with citicoline, especially such liposomes in which phosphate glycerol constitutes 60-90% of the phospholipid external phosphor-containing groups.

The dosage of citicoline in such a combination use is suitably from about 1 to 100 mg per kilogram patient body weight, per administration. The liposomes are suitable administered in a dosage of from about 500 to about 2.5×10¹⁰, prefereably from about 1000 to about 1.5×10⁸, vesicles per dose, in such combinations.

Peripheral vasodilators which can be used in this invention include, but are not limited to: aluminum nicotinate, (U.S. Pat. No. 2,970,082); bamethan, Corrigan et al., Journal of the American Chemical Society, 1945, 67 1894; betahistine, Walter et al.; Journal of the American Chemical Society, 1941, 63 2771; bradykinin, Hamburg et al., Arch. Biochem. Biophys., 1958, 76, 252; brovincamine, (U.S. Pat. No. 4,146,643); bufeniode, (U.S. Pat. No. 3,542,870); buflomedil, (U.S. Pat. No. 3,895,030); butalamine, (U.S. Pat. No. 3,338,899); cetiedil, (French Patent No. 1,460,571); ciclonicate, (German Patent No. 1,910,481); cinepazide, (U.S. Pat. No. 3,634,411); cinnarizine, cyclandelate, diisopropylamine dichloroacetate, eledoisin, (British Patent No. 984,810); fenoxedil, flunarizine, hepronicate, (U.S. Pat. No. 3,384,642); ifenprodil, iloprost, (U.S. Pat. No. 4,692,464); inositol niacinate, Badgelt et al., Journal of the American Chemical Society, 1947, 69, 2907; isoxsuprine, (U.S. Pat. No. 3,056,836); kallidin, (Nicolaides etal., Biochem. Biophys. Res. Commun., 1961, 6, 210); kallikrein, (German Patent No. 1,102,973); moxisylyte, (German Patent No. 905,738); nafronyl, nicametate, nicergoline, nicofuranose, (Swiss Patent No. 366,523); nylidrin, (U.S. Pat. Nos. 2,661,372 and 2,661,373); pentifylline, pentoxifylline, (U.S. Pat. No. 3,422,107); piribedil, (U.S. Pat. No. 3,299,067); prostaglandin E1, suloctidil, (German Patent No. 2,334,404); tolazoline, (U.S. Pat. No. 2,161,938); and xanthinol niacinate, (German Patent No. 1,102,750 or Korbonits et al., Acta. Pharm. Hung., 1968, 38, 98). Each of the above patents and publications is incorporated herein by reference in its entirety.

Diuretics, also sometimes included under the general heading of antihypertensives, include thiazide types, organomercurials, purines, steroids, sulfonamide derivatives, uracils, loop diuretics, potassium sparing diuretics and others. Thiazide types useful in the present invention include, but are not limited to: althiazide, (British Patent No. 902,658); bendroflumethiazide, (U.S. Pat. No. 3,265,573); benzthiazide; benzylhydrochlorothiazide, (U.S. Pat. No. 3,108,097); buthiazide, (British Patent Nos. 861.367 and 885,078); chlorothiazide, (U.S. Pat. Nos. 2,809,194 and 2,937,169); chlorthalidone; cyclopentathiazide; cyclothiazide; ethiazide; fenquizone; hydrochlorothiazide, (U.S. Pat. No. 3,164,588); hydroflumethiazide; indapamide; methycyclothiazide; metalozone; paraflutizide; polythiazide; quinethazon); teclothiazide; and trichloromethiazide. Each of the above patents is incorporated herein by reference in its entirety.

Loop diuretics useful in the present invention include bumetanide, (U.S. Pat. No. 3,634,583); furosemide, (U.S. Pat. No. 3,058,882); and torasemide, (U.S. Pat. No. 4,018,929). Potassium sparing diuretics useful in the invention include triamterene (U.S. Pat. No. 3,081,230). Other useful diuretics for use in the invention are amiloride; chlorazanil; ethacrynic acid; etozolin; isosorbide; mannitol; muzolimine; perhexiline; ticrynafen; triamterene; and urea. Each of the above patents is incorporated herein by reference in its entirety.

Another component of the combination therapy of the present invention is synthetic or semi-synthetic bodies carrying anionic or other phospholipids on their surfaces and having a size from about 20 nanometers to about 500 microns (as measured along its longest axis). The active head groups on the surfaces are sometimes referred to herein as ligands. Such synthetic and semi-synthetic bodies are disclosed below and also found in, for example, Bolton et al., United States Publication Nos. US 2002-0086049, US 2003-0175334, and 2004-0013718, herein incorporated in their entirety by reference.

Examples of three-dimensional body portions include liposomes, solid beads, hollow beads, filled beads, particles, granules and microspheres of biocompatible materials, natural or synthetic, such as polyethylene glycol, polyvinylpyrrolidone, polystyrene, poly(methylmethacrylate), etc., polysaccharides such as hydroxyethyl starch, hydroxyethylcellulose, agarose and the like, as commonly used in the pharmaceutical industry. The beads may be solid or hollow, or filled with biocompatible material.

Suitable substances for derivatisation to attach the phospholipid(s) to three-dimensional bodies are commercially available e.g. from Polysciences Inc., 400 Valley Road, Warrington, Pa. 18976, or from Sigma Aldrich Fine Chemicals.

Preferred compositions of matter are liposomes, which may be composed of a variety of lipids. Liposomes, or lipid vesicles, are sealed sacs, in the micron or sub-micron range, the walls of which comprise suitable amphiphiles. They normally contain an aqueous medium. Generally the liposomes are composed of phosphatidylcholine, phosphatidylglycerol, distearoylphosphatidylcholine, distearoylphosphatidylglycerol, dipalmitoylphosphatidylglycerol, phosphatidylinositol, phosphatidic acid, lysophosphatidic acid, lysophosphatidylinositol, lecithin, cardiolipin, cephalin, cerebrosides including sphingomyelin, and sphingosine. Such liposomes are prepared and treated so that the active polar head groups are presented exteriorly on the liposomal body. Thus a preferred embodiment of this invention provides synthetic or semi-synthetic bodies which expose or can be treated or induced to expose, on their surfaces one or more phospholipid ligands e.g. phosphoamine group with active head groups. These will be found among phosphatidylcholine, phosphatidylglycerol, phosphatidylethanolamine, phosphatidylinositol, phosphatidic acid, lysophosphatidic acid, lysophosphatidylinositol, lysophosphatidylcholine, lysophosphatidylethanolamine, sphingosylphosphoryl choline, sphingosine-1-phosphate, cardiolipin, cholesterol, ceramides, sphingomyelin, some of which are active, and others of which are inactive but provide structure to the liposome.

In the case of such preferred liposomes, the constitution of the liposome may be partially or entirely composed of the active phospholipid, the active head groups disposed on the surface thereof and the lipophilic portion forming the wall or membrane of the liposomal body. At least 10% by weight of such liposome is composed of one or more of the phospholipids having chemically active head groups, preferably at least 50%, more preferably from 60-100% and most preferably from 70-90%, with the single most preferred embodiment being about 75% by weight of active phospholipid. The preferred phospholipids for use in the present invention are phosphatidylglycerol, phosphatidylethanolamine, phosphatidylinositol, cardiolipin and phosphatidic acid, optionally with a minor portion (up to less than 50% by weight) of another, inactive constituent. The most preferred active phospholipid to be used in the present invention is phosphatidylglycerol (PG), even more preferably a liposome constituted to the extent of about 70-90% PG, balance inactive phospholipid such as phosphatidyl-choline.

One particularly preferred liposome mixture constitutes 75% phosphatidylglycerol and 25% phosphatidylcholine.

Analogues of phosphatidylglycerol with modified active head groups, which also interact with the PG receptors on the antigen presenting cells, or otherwise result in an anti-inflammatory reaction in the recipient body, are contemplated within the scope of the term phosphatidylglycerol.

It is also within the scope of the present invention to use bodies having a mixture of the aforementioned phospholipids having chemically active head groups, this mixture comprising at least 10%, preferably at least 50% and most preferably 60-90% of the aforementioned active phospholipids. Instead of the minor constituent being an inactive constituent, it can be active through another mechanism.

Mixtures of liposomes of different aforementioned phospholipids having chemically active head groups, and mixtures of such liposomes with liposomes of inactive and/or with liposomes of phospholipids acting through a different mechanism can also be used, provided that the total amount of active phospholipid remains above the minimum of about 10% and preferably above 50% in the total mixture.

It is postulated that, in many embodiments of the present invention, synthetic or semi-synthetic bodies carrying anti-inflammatory promoting ligands on their surface are acting as modifiers of the patient's immune system, in a manner similar to that of a vaccine. Accordingly they are preferably used in quantities and by administration methods to provide a sufficient localized concentration of the bodies at the site of introduction. Quantities of synthetic or semi-synthetic bodies carrying on their surfaces anti-inflammatory promoting ligands, appropriate for immune system modifying substances are generally not directly correlated with body size of a recipient and can, therefore, be clearly distinguished from drug dosages, which are designed to provide therapeutic levels of active substances in a patient's bloodstream and tissues. Drug dosages are, accordingly, likely to be much larger than immune system modifying dosages.

It is contemplated that the patient may be a mammal, including but not limited to humans and domestic animals such as cows, horses, pigs, dogs, cats and the like.

Phospholipids are amphiphilic molecules (i.e. amphiphiles), meaning that the compound comprises molecules having a polar water-soluble group attached to a water-insoluble hydrocarbon chain. The amphiphiles serving as the layers of the matrix have defined polar and apolar regions. The amphiphiles can include, in addition to the phospholipids providing the active head groups in the process of the invention, other, naturally occurring lipids used alone with the phospholipid carrying the active head group, or in a mixture with another. The amphiphiles serving as the layer(s) of the liposomes can be inert, structure-conferring synthetic compounds such as polyoxyethylene alkylethers, polyoxyethylene alkylesters and saccharosediesters.

Methods of preparing liposomes of the appropriate size are known in the art and do not form part of this invention. Reference may be made to various textbooks and literature articles on the subject, for example, the review article “Liposomes as Pharmaceutical Dosage Forms”, by Yechezkel Barenholz and Daan J. A. Chrommelin, and literature cited therein, for example New, R. C. “Liposomes: A Practical Approach”, IRL Press at Oxford University Press (1990).

The diameter of the ligand-carrying liposomes of the preferred embodiment of this invention is from about 20 nanometers to about 500 microns, more preferably from about 20 nanometers to about 1000 nanometers, more preferably from about 50 nanometers to about 500 nanometers, and most preferably from about 80 nanometers to about 120 nanometers. Such preferred diameters will generally correspond to the diameters of mammalian apoptotic bodies or apoptotic cells. Each of these diameters is measured along its longest axis.

The ligand-carrying bodies may be suspended in a pharmaceutically acceptable carrier, such as physiological sterile saline, sterile water, pyrogen-free water, isotonic saline, and phosphate buffer solutions, as well as other non-toxic compatible substances used in pharmaceutical formulations. Preferably, the ligand-carrying bodies are constituted into a liquid suspension in a biocompatible liquid such as buffered saline and administered to the patient in any appropriate route which introduces it to the immune system, such as intra-arterially, intravenously or most preferably intramuscularly or subcutaneously.

It is contemplated that the ligand-carrying bodies may be freeze-dried or lyophilized so that they may be later resuspended for administration. This invention is also directed to a kit of part comprising lyophilized or freeze-dried ligand-carrying bodies and a pharmaceutically acceptable carrier, such as physiological sterile saline, sterile water, pyrogen-free water, isotonic saline, and phosphate buffer solutions, as well as other non-toxic compatible substances used in pharmaceutical formulations.

A preferred manner of administering the ligand-carrying bodies to the patient is a course of injections, administered daily, several times per week, weekly or monthly to the patient, over a period ranging from a week to several months. The frequency and duration of the course of the administration is likely to vary from patient to patient, and according to the condition being treated, its severity, and whether the treatment is intended as prophylactic, therapeutic or curative. Its design and optimization is well within the skill of the attending physician. Intramuscular injection, especially via the gluteal muscle, is most preferred. One particular injection schedule, in at least some of the indication of the invention, is an injection, via the gluteal muscle, of an appropriate amount of bodies on day 1, a further injection on day 2, a further injection on day 14, and then “booster” injections at monthly intervals.

The quantities of ligand-carrying bodies to be administered will vary depending on the nature of the mammalian disorder it is intended to treat and on the identity and characteristics of the patient. It is important that the effective amount of ligand-carrying bodies is non-toxic to the patient, and is not so large as to overwhelm the immune system. When using intra-arterial, intravenous, subcutaneous or intramuscular administration of a liquid suspension of ligand-carrying bodies, it is preferred to administer, for each dose, from about 0.1-50 ml of liquid, containing an amount of ligand-carrying bodies generally equivalent to 10% -1000% of the number of leukocytes normally found in an equivalent volume of whole blood or the number of apoptotic bodies that can be generated from them. Generally, the number of ligand-carrying bodies administered per delivery to a human patient is in the range from about 500 to about 2.5×10⁹ (<250 ng of bodies, in the case of liposomes, pro-rated for density differences for other embodiments of bodies), more preferably from about 1,000 to about 1,500,000,000, even more preferably 10,000 to about 100,000,000, and most preferably from about 200,000 to about 2,000,000.

Since the ligand-carrying bodies are acting, in the process of the invention, as immune system modifiers, in the nature of a vaccine, the number of such bodies administered to an injection site for each administration is a more meaningful quantitation than the number or weight of ligand-carrying bodies per unit of patient body weight. For the same reason, it is contemplated that effective amounts or numbers of ligand-carrying bodies for small animal use may not

1. A process for alleviating the symptoms of a cardiovascular disorder in a mammalian patient, which comprises administration to the patient
a) an effective amount of synthetic and/or semi-synthetic bodies carrying phospholipid ligands on their surfaces, said bodies having a size of from about 20 nanometers to about 500 microns; and
b) an effective amount of a cardiovascular drug.

2. A process according to claim 1 wherein the cardiovascular drug is selected from the group consisting of an antihypertensive drug, an anti-anginal drug, an anti-arrhythmia drug, an antihyperlipoproteinemia drug, a calcium channel blocker, a cardiotonic drug, a coronary vasodilator, a peripheral vasodilator, a cerebral vasodilator and a diuretic.

3. A process according to claim 1 wherein the cardiovascular drug is an antihypertensive drug selected from the group consisting of ACE-inhibitors, alpha-adrenergic agonists, beta-adrenergic agonists, alpha-adrenergic blocker, beta-adrenergic blockers, and angiotensin II receptor antagonists.

4. A process according to claim 1 wherein the cardiovascular drug is an antihyperlipoproteinemia drug.

5. A process according to claim 4 wherein the antihyperlipoproteinemia drug is a statin.

6. A process according to claim 5 wherein the statin is atorvastatin or atorvastatin calcium.

7. A process according to claim 1 wherein the phospholipid ligands are phosphatidylglycerol.

8. A composition for treating a cardiovascular disorder in a mammalian patient, said composition comprising (a) an effective amount of synthetic and/or semi-synthetic bodies carrying phospholipid ligands on their surfaces, said bodies having a size of from about 20 nanometers to about 500 microns, and (b) an effective amount of a cardiovascular drug.

9. A process according to claim 1 wherein the cardiovascular disorder is selected from the group consisting of atherosclerosis, hypertension, arrhythmia, angina, chronic heart failure, and peripheral arterial occlusive diseases.

10. A process according to claim 2 wherein the drug is carvedilol.

11. A process according to claim 10 wherein the synthetic or semi-synthetic bodies are liposomes comprising phosphatidylglycerol with externalised phosphate-glycerol groups.

12. A process according to claim 2 wherein the drug is citicoline.

13. A process according to claim 12 wherein the synthetic or semi-synthetic bodies are liposomes comprising phosphatidylglycerol with externalised phosphate-glycerol groups.

14. A composition for treating vascular disorder in a mammalian patient, said composition comprising (a) an effective amount of synthetic and/or semi-synthetic bodies carrying phospholipid ligands on their surfaces, said bodies having a size of from about 20 nanometers to about about 500 microns, and (b) an effective amount of a drug selected from the group consisting of a citicoline and carvedilol.

15. The composition of claim 14 wherein the drug is citicoline.
